(19) United States
(12) Patent Application Publication
(10) Pub. No.: US 2005/0247321 A1
(43) Pub. Date: Nov. 10, 2005

(54) Laser perforator

(76) Inventors: Milton Waner, Little Rock, AR (US); Charles H. Vestal, Little Rock, AR (US); Stephen T. Flock, Edmonton (CA)

Correspondence Address: Dr. Benjamin Adler;Adler & Associates, 8011 Candle Lane, Houston, TX, 77071, US

(21) Appl. No.: 11/014,352

(22) Filed: Dec. 16, 2004

Related U.S. Application Data
(63) Continuation of application No. 10/295,102, filed on Nov. 15, 2002, now abandoned.
Application No. 10/295,102 is a continuation of application No. 09/447,492, filed on Nov. 23, 1999, now abandoned.
Application No. 09/447,492 is a continuation of application No. 08/686,418, filed on Jul. 26, 1996, now abandoned.
Application No. 08/686,418 is a continuation-in-part of application No. 08/126,241, filed on Sep. 24, 1993, now Pat. No. 5,643,252.
Application No. 08/126,241 is a continuation-in-part of application No. 07/968,862, filed on Oct. 28, 1992, now abandoned.

(51) Int. Cl. A61B 19/00; A61N 1/30; A61B 18/18

(52) U.S. Cl. 128/898; 606/9; 604/20

(57) ABSTRACT

The present invention provides methods of introducing a substance into a living body by forming a plurality of areas on the stratum corneum of the skin such that each area has an enhanced permeability through to the capillary layer. A single laser beam is generated to irradiate the areas on the skin with subablative laser energy to perforate the skin to specific depths without substantially ablating the skin. The substance is introduced into the body by bringing the substance in contact with the areas of enhanced permeability. The present invention further provides lasers comprising a crystal with a partial matte surface-mirror element in combination with a beam splitter to generate a plurality of laser beams substantially concurrently from the single laser beam or with an acousto-optical modulator to sequentially deflect the single laser beam at a plurality of angles.

This application is a continuation of pending U.S. Ser. No. 10/295,102, filed Nov. 15, 2002, which is a continuation of U.S. Ser. No. 09/447,492, filed Nov. 23, 1999, now abandoned, which is a continuation of U.S. Ser. No. 08/686,418, filed Jul. 26, 1996, now abandoned, which is a continuation-in-part of U.S. Ser. No. 08/126,241, filed Sep. 24, 1993, now issued as U.S. Pat. No. 5,643,252 on Jul. 1, 1997, which is a continuation-in-part of U.S. Ser. No. 07/968,862, filed Oct. 28, 1992, now abandoned, all of which applications are incorporated herein by reference.

### FIELD OF THE INVENTION

This invention is in the field of medical equipment, namely laser medical equipment.

### BACKGROUND

The traditional method for the collection of small quantities of blood from a patient utilizes mechanical perforation of the skin with a sharp device such as a metal lancet or needle. This procedure has many drawbacks, two of which are the possible infection of health-care workers or the public at large with the device used to perforate the skin, and the costly handling and disposal of biologically hazardous waste.

When skin is perforated with a sharp device such as a metal lancet or needle, biological waste is created in the form of the “sharp” which is contaminated by the patient's blood and/or tissue. If the patient is infected with any number of blood-born agents, such as human immunodeficiency virus (HIV) which causes autoimmune deficiency syndrome (AIDS), hepatitis virus, or the etiological agent of other diseases, the contaminated sharp can pose a serious threat to others who might come in contact with it. There are many documented instances of HIV infection of medical workers who were accidentally stabbed by a contaminated sharp.

Disposal of sharps is also a major problem. Disposal of contaminated materials poses both a logistics and a financial burden on the end user such as the medical institution. In the 1980s, numerous instances of improperly disposed biological wastes being washed up on public beaches have occurred. The potential for others, such as intravenous drug users, to obtain improperly disposed needles is also problematic.

There exists an additional drawback using the traditional method of being stabbed by a sharp instrument for the purpose of drawing blood. Often, the stabbing procedure must be repeated before sufficient blood is obtained. This can cause significant stress and anxiety in the patient.

Clearly, the current procedure for puncturing skin for the purpose of drawing blood has significant inherent problems. These problems arise because a sharp instrument is used in the procedure. Thus, a need exists for a technique to puncture skin which does not use a sharp instrument. This method would obviate the need for disposal of contaminated instruments, and reduce the risk of cross infection.

Lasers have been used in recent years as a very efficient precise tool in a variety of surgical procedures. Among potentially new sources of laser radiation, the rare-earth elements are of major interest for medicine. The most promising of these is a YAG (yttrium, aluminum, garnet) crystal doped with erbium (Er) ions. With the use of this crystal, it is possible to build an erbium-YAG (Er:YAG) laser which can be configured to emit electromagnetic energy at a wavelength (2.94 microns) which is strongly absorbed by water. When tissue, which consists mostly of water, is irradiated with radiation at or near this wavelength, it is rapidly heated. If the intensity of the radiation is sufficient, the heating is rapid enough to cause the vaporization of tissue. Some medical uses of Er:YAG lasers have been described in the health care disciplines of dentistry, gynecology and ophthalmology (Bogdasarov, B. V., et al., “The Effect of YAG:Er Laser Radiation on Solid and Soft Tissues,” Preprint 266, Institute of General Physics, Moscow, 1987; Bolshakov, E. N. et al., “Experimental Grounds for YAG:Er Laser Application to Dentistry,” SPIE 1353:160-169, Lasers and Medicine (1989)). Er:YAG lasers, along with other solid state lasers often employ a polished barrel crystal element such as a polished rod. A laser built with such a polished element maximizes the laser's energy output. Other lasers employ an entirely frosted element, normally with matte of about 50-55 microinch. However, in both cases, the energy output is typically separated into a central output beam surrounded by halo rays or has an otherwise undesirable mode. Since it is extremely difficult to focus halo rays to a specific spot the laser output may be unacceptable for specific applications.

Solid state lasers also typically employ two optic elements in connection with the crystal element. The optic elements consist of the rear high reflectance mirror and the front partial reflectance mirror, also know as an output coupler. The crystal element and the optic elements are rigidly mounted in order to preserve the alignment between them. However, changes in temperature, such as that caused by expansion of the crystal rod during flash lamp exposure, also cause shifts in alignment between the mirrors and the crystal. The misalignment of the mirrors and the crystal element results in laser output energy loss. Thus, the rigidly mounted elements require constant adjustment and maintenance. Moreover, thermal expansion of the crystal element during lasing can cause the crystal to break while it is rigidly attached to a surface with different expansion characteristics.

### SUMMARY OF THE INVENTION

The present invention eliminates the halo ring of a solid state laser's output by creating a surface finish of the crystal element which is matte only over part of its surface. The present invention also employs mirrored surfaces applied to the ends of the crystal element, such as in the form of coatings which reflect light at the desired wavelengths. By making the crystal and mirrors part of the same element the invention eliminates the problems associated with misalignment of the element and the mirrors.

Combining the mirrors and crystal in a single element permits non-rigid mounting of the crystal element. Thus, the present invention employs the crystal-mirror element mounted by means of an elastomeric material. The elastomeric material provides a self-centering action to properly position the element so as to capture the light energy from the laser excitation source while allowing for movement of the element due to thermal expansion. The foregoing inventions may be applied to all solid state lasers.

The foregoing inventions may also be employed with a laser utilized to perforate the skin of a patient. In a separate invention perforation of a patient's skin is produced by irradiating the surface of the skin by a focused pulse of electromagnetic energy emitted by a laser. It is possible to very precisely perforate skin to a selectable depth without causing clinically relevant damage to healthy proximal tissue by a judicious selection of the following irradiation parameters: wavelength, energy fluence (determined by dividing the energy of the pulse by the area irradiated), pulse temporal width and irradiation spot size.

A device is provided which emits a pulsed laser beam, focused to a small spot for the purpose of perforating tissue. By adjusting the output of the laser, the depth, width and length of the perforation can be controlled to fit the purpose for which the perforation is required. This method can be used to create a small, relatively shallow hole in the skin which penetrates into the capillary bed, thus allowing the drawing of blood for a variety of purposes. Optionally a tissue pre-heating device can be added to increase blood flow prior to the laser perforation. Safety interlocks are advantageously incorporated in the device to prevent hazardous operation and accidental laser-irradiations.

This device can further be modified to include a container unit. Such a container unit can be added to: (1) increase the efficiency in the collection of blood and serum; (2) reduce the noise created when the laser beam perforates the patient's tissue; and (3) collect the ablated tissue. The container unit is optionally evacuated to expedite the collection of blood and serum. In one embodiment, the container unit collects only ablated tissue. The noise created from the laser beam's interaction with the patient's skin may cause the patient anxiety. The optional container unit reduces the noise intensity and therefore alleviates the patient's anxiety and stress. The container unit also minimizes the risk of cross-contamination and guarantees the sterility of the collected sample. The placement of the container unit in the device of this invention is unique in that it covers the tissue being punctured, at the time of puncture by the laser beam, and is therefore able to collect the blood sample and/or ablated tissue as the puncturing occurs.

This invention also provides a means for puncturing the skin of a patient in a manner that does not result in bleeding. The perforation created typically penetrates through the keratin layer or both the keratin layer and the epidermis. This will allow the administration of pharmaceuticals through the skin. There are several advantages to administering drugs in this fashion, for example: drugs can be administered continually on an out-patient basis over long periods of time, and the speed and/or efficiency of drug delivery can be enhanced for drugs which were either slow or unable to penetrate skin. Furthermore, this method of delivery provides an alternative delivery route for drugs that would otherwise require to be injected.

This invention avoids the use of sharps. The absence of a contaminated sharp will eliminate the risk of accidental injury and its attendant risks to the healthcare worker, the patient, and anyone who may come into contact with the sharp, whether by accident or by necessity.

The absence of sharps also obviates the need for disposal of biologically hazardous waste. Thus, this invention provides an ecologically sound method of perforating skin.

The device of this invention requires no special skills to use. It is small, light-weight and can be used with rechargeable batteries. This portability and ease of use makes possible the utility of the device in a variety of settings, such as a hospital room, clinic, or home.

The safety features incorporated into this device do not require that any special safety eyewear be worn by the operator of the device, the patient, or anyone else in the vicinity of the device when it is being used. This is a marked improvement over prior art laser devices which require such special protection.

### BRIEF DESCRIPTION OF THE DRAWINGS

The present invention may be better understood and its advantages appreciated by those skilled in the art by referring to the accompanying drawings wherein

FIG. 1 shows the laser device with its power source, high voltage pulse-forming network, flashlamp, lasing rod, mirrors, housing and focusing lens.

FIG. 2 shows an optional spring-loaded interlock and optionally heated applicator.

FIG. 3 shows an alternative means of exciting the laser rod using a diode laser.

FIG. 4 shows an alternative focusing mechanism.

FIG. 5A-5B show optional beam splitters for creating multiple simultaneous perforations.

FIG. 6 shows a patch which can be used to sterilize the perforation site.

FIG. 7A-7B show alternative patches for sterilization and/or delivery of pharmaceuticals.

FIG. 8 shows an optional container unit for collecting blood, ablated tissue, and/or other matter released from the perforation, and for reducing noise resulting from the interaction between the laser and the patient's tissue.

FIG. 9 shows a plug and plug perforation center.

FIG. 10 shows an optional container unit for collecting ablated tissue and reducing noise resulting from the interaction between the laser and the patient's tissue.

FIG. 11 shows an optional version of the collection container unit which is especially useful when the container unit includes a reagent for mixing with the sample.

FIG. 12 shows an elastomeric mount for a solid state laser crystal element with optional mirrored surfaces applied to each end of the element.

FIG. 13 shows an example of a crystal rod with matte finish around the full circumference of the entire rod.

FIG. 14 shows an example of a crystal rod with matte finish around the full circumference of two-thirds of the rod.

FIG. 15 shows an example of a crystal rod with matte stripes along its longitudinal axis.

FIG. 16 shows a cross-section of a crystal laser rod element surrounded by a material having an index of refraction greater than the index of refraction of the rod.

FIG. 17 shows various examples of the container unit.

FIG. 18 shows the container unit with the additional vessel.

FIG. 19 shows examples of the container unit in use with the laser perforator device.

FIG. 20 shows an example of a lens with a mask.

### DETAILED DESCRIPTION OF THE PREFERRED EMBODIMENTS

This invention provides a method and device for perforating skin for either the sampling of blood or the administration of pharmaceuticals. The device utilizes a laser beam, specifically focused, and lasing at an appropriate wavelength, preferably between 2 and 7 microns, to create small holes in the skin of a patient. The laser beam is focused with a lens to produce an irradiated spot on the skin with a size of approximately 0.1-1 mm diameter, and an energy fluence in the range of 10-100,000 J/cm.sup.2. Optionally, the spot can be slit-shaped, with a width of 0.05-0.5 mm and a length of up to 2.5 mm.

The Device

As shown in the Figures, the device comprises a power connection which can be either a standard electrical supply 10, or optionally a rechargeable battery pack 12, optionally with a power interlock switch 14 for safety purposes; a high voltage pulse-forming network 16; a laser pump-cavity 18 containing a laser rod 20, preferably Er:YAG; a means for exciting the laser rod, preferably a flashlamp 22 supported within the laser pump-cavity; an optical resonator comprised of a high reflectance mirror 24 positioned posterior to the laser rod and an output coupling mirror 26 positioned anterior to the laser rod; a transmitting focusing lens 28 positioned beyond the output coupling mirror; optionally a second focusing cylindrical lens 27 positioned between the output coupling mirror and the transmitting focusing lens; an applicator 30 for positioning the subject skin at the focal point of the laser beam, which is optionally heated for example with a thermoelectric heater 32, attached to the laser housing 34; an interlock 36 positioned between the applicator and the power supply; and optionally a beam dump 38 attached to the applicator with a fingertip access port 40.

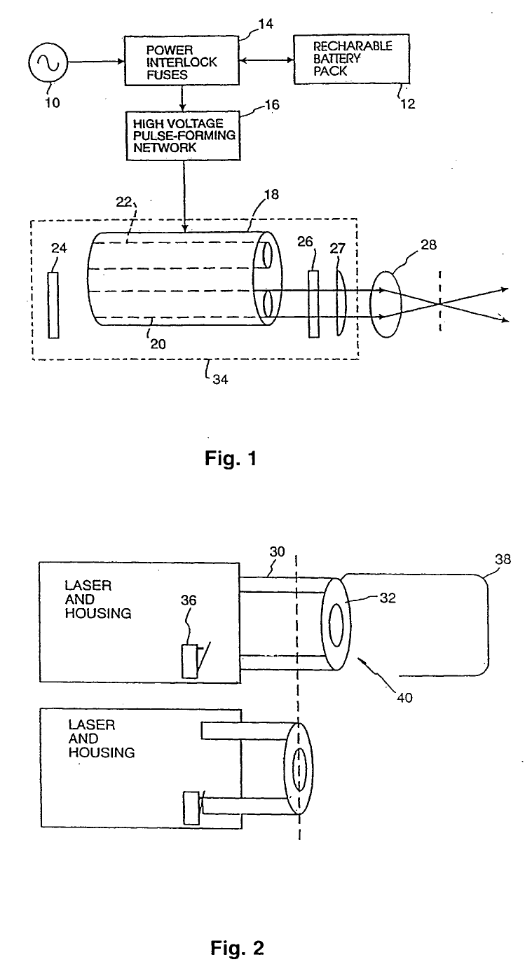

FIGS. 1-2 are diagrammatic representations of the preferred embodiment of the device of the invention. The device preferably draws power from a standard 110 V or 220 V line 10 (single phase, 50 or 60 Hz) which is rectified and used to charge up a bank of capacitors included in the high voltage pulse-forming network 16. Optionally, a rechargeable battery pack 12 can be used instead. The bank of capacitors establishes a high DC voltage across a high-output flashlamp 22. Optionally a power interlock 14, such as a keyswitch, can be provided which will prevent accidental charging of the capacitors and thus accidental laser excitation. A further interlock can be added to the device at the applicator, such as a spring-loaded interlock 36, so that discharge of the capacitors requires both interlocks to be enabled.

With the depression of a switch, a voltage pulse can be superimposed on the already existing voltage across the flashlamp in order to cause the flashlamp to conduct, and, as a consequence, initiate the flash. The light from the flashlamp is located in the laser cavity 18 that has a shape such that most of the light is efficiently directed to the laser rod 20, which absorbs the light, and, upon de-excitation, subsequently lases. The laser cavity mirrors of low 26 and high 24 reflectivity, positioned collinearly with the long-axis of the laser rod, serve to amplify and align the laser beam.

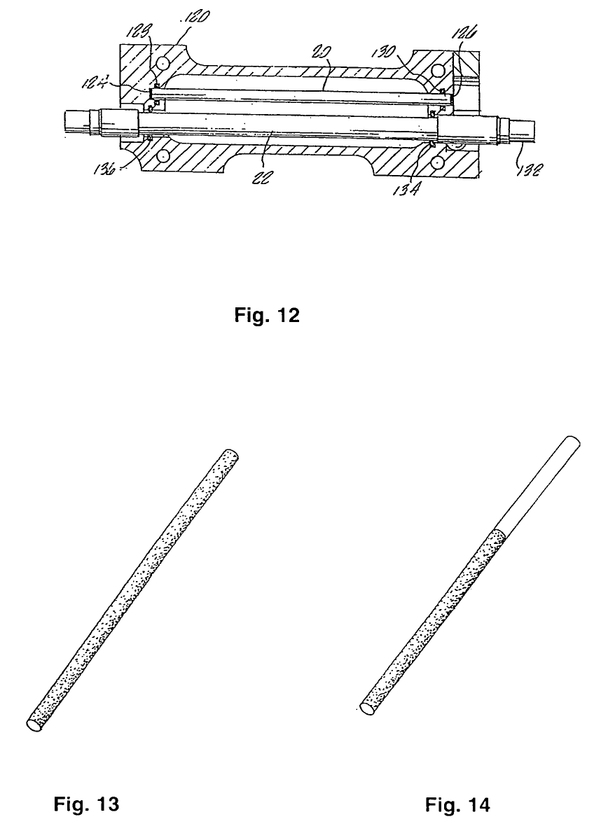

Optionally, as shown in FIG. 12 the laser cavity mirrors comprise coatings 124, 126, applied to ends of the crystal element and which have the desired reflectivity characteristics. In a preferred embodiment an Er:YAG crystal is grown in a boule two inches in diameter and five inches long. The boule is core drilled to produce a rod 5-6 millimeters in diameter and five inches long. The ends of the crystal are ground and polished. The output end, that is the end of the element from which the laser beam exits, is perpendicular to the center axis of the rod within 5 arc minutes. The flatness of the output end is 1/10 a wavelength (2.9 microns) over 90% of the aperture. The high reflectance end, that is the end opposite the output end, comprises a two meter convex spherical radius. The polished ends are polished so that there are an average of ten scratches and five digs per Military Specification Mil-0-13830A. Scratch and dig are subjective measurements that measure the visibility of large surface defects, such as defined by U.S. military standards. Ratings consist of two numbers, the first being the visibility of scratches and the latter being the count of digs (small pits). A #10 scratch appears identical to a 10 micron wide standard scratch while a #1 dig appears identical to a 0.01 mm diameter standard pit. For collimated laser beams, one normally would use optics with better than a 40-20 scratch-dig rating.

Many coatings are available from Rocky Mountain Instruments, Colorado Springs, Colo. The coating is then vacuum deposited on the ends. For a 2.9 micron wavelength the coatings for the rear mirrored surface 124 should have a reflectivity of greater than 99%. The coating for the output end surface, by contrast, should have a reflectance of between 93% and 95%. Other vacuum deposited metallic coatings with known reflectance characteristics are widely available for use with other laser wavelengths. The general equation which defines the reflectivity of the mirrors in a laser cavity necessary for the threshold for population inversion is:
R.sub.1R.sub.2(1−a.sub.L).sup.2exp[(g.sub.21,−.alpha.)2L]=1;
where the R.sub.1 and R.sub.2 are the mirrors'reflectivities, a.sub.L is the total scattering losses per pass through the cavity, g.sub.21 is the gain coefficient which is the ratio of the stimulated emission cross section and population inversion density, a is the absorption of the radiation over one length of the laser cavity, and L is the length of the laser cavity. Using the above equation, one can select a coating with the appropriate spectral reflectivity from the following references. Handbook of Optics, ch. 8, (W. Driscoll and W. Vaughan eds., McGraw-Hill: N.Y. 1978); 1 Handbook of Optics, ch. 35, (M. Bass, et al., eds., McGraw Hill: N.Y. 1995).

Optionally, as also shown in FIG. 12, the crystal element may be non-rigidly mounted. In FIG. 12 an elastomeric material O-ring 128 is in a slot in the laser head assembly housing 120 located at the high reflectance end of the crystal element. A second elastomeric material O-ring 130 is in a second slot in the laser head assembly at the output end of the crystal element. The O-rings contact the crystal element by concentrically receiving the element as shown. However, elastomeric material of any shape may be used so long as it provides elastomeric support for the element (directly or indirectly) and thereby permits thermal expansion of the element. Optionally, the flash lamp 22 may also be non-rigidly mounted. FIG. 12 shows elastomeric O-rings 134, 136, each in its own slot within the laser head assembly housing. In FIG. 12 the O-rings 134 and 136 concentrically receive the flash lamp. However, the flash lamp may be supported by elastomeric material of other shapes, including shapes without openings.

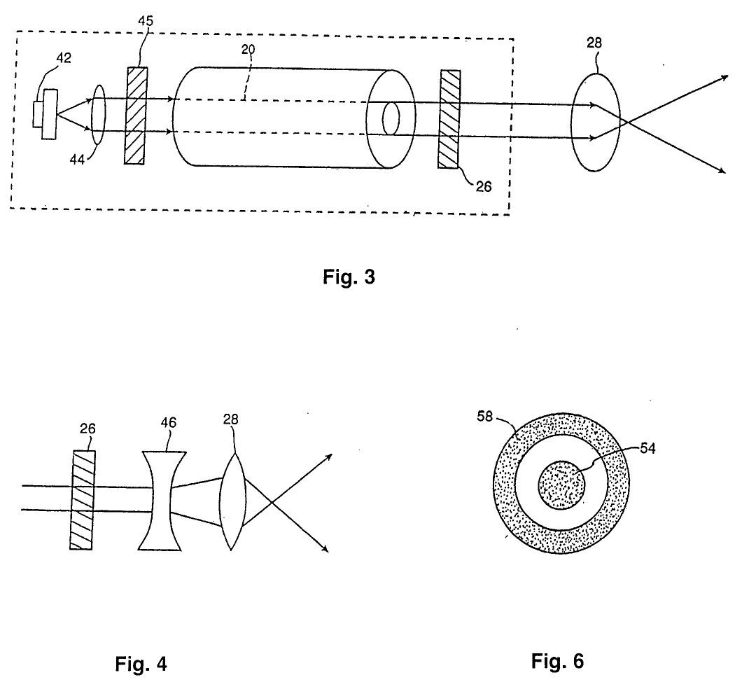

Optionally, as shown in FIG. 3, a diode laser 42 which produces a pump-beam collinear with the long-axis of the laser crystal can be used instead of the flashlamp to excite the crystal. The pump-beam of this laser is collimated with a collimating lens 44, and transmitted to the primary laser rod through the high reflectance infrared mirror 45. This high reflectance mirror allows the diode pump laser beam to be transmitted, while reflecting info light from the primary laser.

The Er:YAG lasing material is the preferred material for the laser perforator rod because the wavelength of the electromagnetic energy emitted by this laser, 2.94 microns, is very near one of the peak absorption wavelengths (approximately 3 microns) of water. Thus, this wavelength is strongly absorbed by water and tissue. The rapid heating of water and tissue causes perforation of the skin.

Other useful lasing material is any material which, when induced to lase, emits a wavelength that is strongly absorbed by tissue, such as through absorption by water or nucleic acids or proteins, and consequently causes the required perforation of the skin. A laser can effectively cut tissue to create the desired perforations where tissue exhibits an absorption coefficient of 10-10,000 cm.sup.−1. Examples of useful lasing elements are pulsed CO.sub.2 lasers, Ho:YAG (holmium:YAG), Er:YAP, Er/Cr:YSGG (erbium/chromium: yttrium, scandium, gallium, garnet; 2.796 microns), Ho:YSGG (holmium: YSGG; 2.088 microns), Er:GGSG (erbium: gadolinium, gallium, scandium, garnet), Er:YLF (erbium: yttrium, lithium, fluoride; 2.8 microns), Tm:YAG (thulium: YAG; 2.01 microns), Ho:YAG (holmium: YAG; 2.127 microns); (cobalt: magnesium fluoride; 1.75-2.5 microns), HF chemical (hydrogen fluoride; 2.6-3 microns), DF chemical (deuterium fluoride; 3.6-4 microns), carbon monoxide (5-6 microns), deep UV lasers, and frequency tripled Nd:YAG (neodymium:YAG, where the laser beam is passed through crystals which cause the frequency to be tripled).

Utilizing current technology, some of these laser materials provide the added benefit of small size, allowing the laser perforator device to be small and portable. In addition to Er:YAG, Ho:YAG lasers provide this advantage.

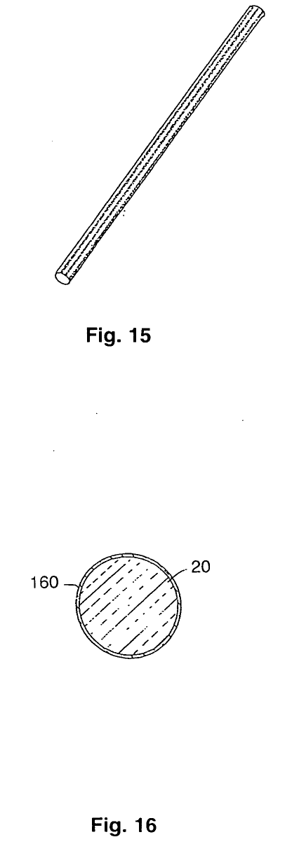

Solid state lasers, including but not limited to those listed above, may employ a polished barrel crystal rod. The rod surface may also contain a matte finish as shown in FIG. 13. However, both of these configurations can result in halo rays which surround the central output beam. Furthermore, an all-matte finish, although capable of diminishing halo rays relative to a polished rod, will cause a relatively large decrease in the overall laser energy output. In order to reduce halo rays and otherwise affect beam mode the matte finish of the instant invention is present on bands of various lengths along the rod, each band extending around the entire circumference of the rod. Alternatively, the matte finish may be present in bands along only part of the rod's circumference. In a preferred embodiment FIG. 14 shows a laser crystal element 142 in which the matte finish is present upon the full circumference of the element along two-thirds of its length. Alternatively, as shown in FIG. 15, matte stripes may be present longitudinally along the full length of the rod. The longitudinal stripes may alternatively exist along only part of the length of the rod, such as in stripes of various lengths. A combination of the foregoing techniques may be used to affect beam shape. Other variations of patterns may also be employed in light of the beam shape desired. The specific pattern may be determined based on the starting configuration of the beam from a 100% polished element in light of the desired final beam shape and energy level. A complete matte finish element may also be used as the starting reference point.

For purposes of beam shape control, any surface finish of greater than 30 microinch is considered matte. A microinch equals one millionth (0.000001) inch. This is a common unit of measurement employed in establishing standard roughness unit values. The degree of roughness is calculated using the root-mean-square average of the distances in microinches above or below the mean reference line, by taking the square root of the mean of the sum of the squares of these distances. Although matte surfaces of greater than 500 microinch may be used to affect beamshape such a finish will seriously reduce the amount of light energy that enters the crystal rod and thus cut the laser's energy.

In one preferred embodiment, designed to remove the beam halo, a matte area of approximately 50 microinch is present around the full circumference of an Er:YAG laser rod for two-thirds the length of the rod. The non-matte areas of the rod are less than 10 microinch. A baseline test of the non-matte rod can be first conducted to determine the baseline beam shape and energy of the rod. The matte areas are then obtained by roughing the polished crystal laser rod, such as with a diamond hone or grit blaster. The specific pattern of matte can be determined with respect to the desired beam shape and required beam energy level. This preferred embodiment results in a greatly reduced beam halo. The rod may also be developed by core drilling a boule of crystal so that it leaves an overall matte finish and then polishing the desired areas, or by refining a partially matte, partially polished boule to achieve the desired pattern.

The beam shape of a crystal laser rod element may alternatively be modified as in FIG. 16 by surrounding the rod 20 in a material 160 which is transparent to the exciting light but has an index of refraction greater than the rod. Such an embodiment can reduce the halo of the beam by increasing the escape probability of off-axis photons within the crystal. This procedure may be used in place or in addition to the foregoing matte procedure.

The emitted laser beam is focused down to a millimeter or submillimeter sized spot with the use of the focusing lens 28. Consideration of laser safety issues suggests that a short focal length focusing lens be used to ensure that the energy fluence rate (W/cm.sup.2) is low except at the focus of the lens where the tissue sample to be perforated is positioned. Consequently, the hazard of the laser beam is minimized.

The beam can be focused so that it is narrower along one axis than the other in order to produce a slit-shaped perforation through the use of a cylindrical focusing lens 27. This lens, which focuses the beam along one axis, is placed in series with the transmitting focusing lens 28. When perforations are slit-shaped the pain associated with the perforation is considerably reduced.

Optionally, the beam can be broadened, for instance through the use of a concave diverging lens 46 (see FIG. 4), prior to focusing through the focusing lens 28. This broadening of the beam results in a laser beam with an even lower energy fluence rate a short distance beyond the focal point, consequently reducing the hazard level. Furthermore, this optical arrangement reduces the optical aberrations in the laser spot at the treatment position, consequently resulting in a more precise perforation.

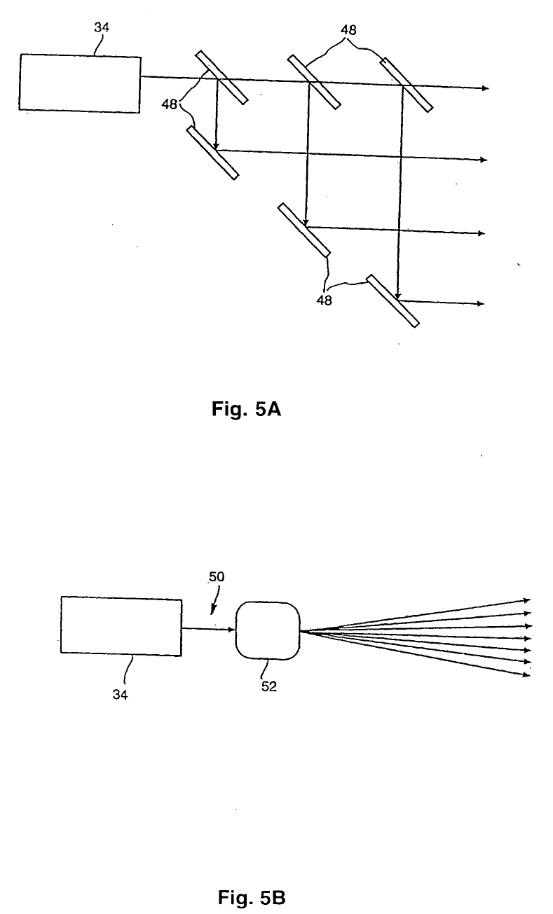

Also optionally, the beam can be split by means of a beam-splitter to create multiple beams capable of perforating at several sites simultaneously or near simultaneously. FIGS. 5A-5B provide two variations of useful beam splitters. In one version, multiple beam splitters 48 such as partially silvered mirrors, dichroic mirrors, or beam-splitting prisms can be provided after the beam is focused. Alternatively, an acousto-optic modulator 52 can be supplied with modulated high voltage to drive the modulator 52 and bend the beam. This modulator is outside the laser cavity. It functions by deflecting the laser beam sequentially and rapidly at a variety of angles to simulate the production of multiple beams.

A small heater, such as a thermoelectric heater 32, is optionally positioned at the end of the laser applicator proximal to the site of perforation. The heater raises the temperature of the skin and capillaries in the tissue to be perforated prior to laser irradiation. This increases blood flow, which increases the volume of blood collected when the device is used for that purpose. A suggested range for skin temperature is between 36.degree. C. and 45.degree. C., although any temperature which causes vasodilation and the resulting increase in blood flow without altering the blood chemistry is appropriate.

A container unit 68 is optionally fitted into the laser housing and is positioned proximal to the perforation site. The container unit reduces the intensity of the sound produced when the laser beam perforates the patient's tissue, increases the efficiency of blood collection, and collects the ablated tissue and other matter released by the perforation. The container unit is shaped so as to allow easy insertion into the laser housing and to provide a friction fit within the laser housing. FIG. 8 shows the container unit inserted into the laser housing and placed over the perforation site.

The container unit 68 comprises a main receptacle 82, including a lens 84. The main receptacle collects the blood sample, the ablated tissue, and/or other matter released by the perforation. The lens is placed such that the laser beam may pass through the lens to the perforation site but so that the matter released by the perforation does not splatter back onto the applicator. The container unit also optionally includes a base 86, attached to the receptacle. The base can optionally be formed so at to be capable of being inserted into the applicator to disengage a safety mechanism of the device, thereby allowing the laser beam to be emitted.

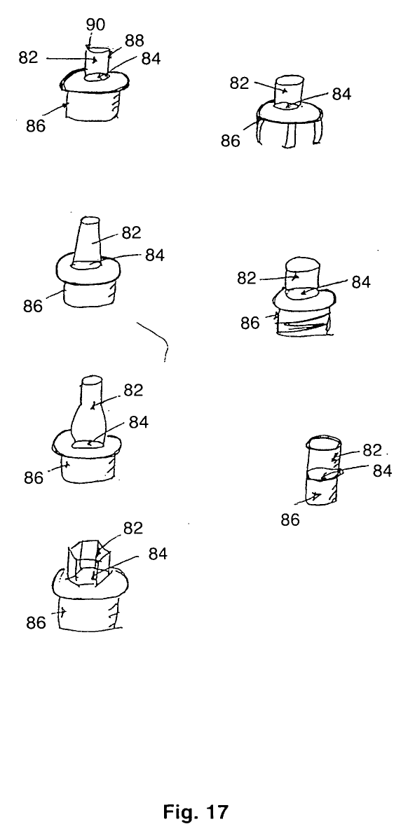

As shown in FIG. 17, the shape and size of the container unit 68 are such as to allow placement next to or insertion into the applicator, and to allow collection of the blood sample, ablated tissue, and/or other matter released by the perforation. Examples of shapes that the main receptacle may take include cylinders, bullet shapes, cones, polygons and free form shapes. Preferably, the container unit has a main receptacle, with a volume of around 1-2 milliliters. However, larger and smaller receptacles will also work.

The lens 84, which allows the laser beam to pass through while preventing biological and other matter from splattering back onto the applicator, is at least partially transparent. The lens is constructed of a laser light-transmitting material and is positioned in the pathway of the laser beam, at the end of the container unit proximal to the beam. In one embodiment, the transmitting material is quartz, but other examples of suitable infrared materials include rock salt, germanium, and polyethylene. As shown in FIG. 20, the lens may optionally include a mask of non-transmitting material 85 such that the lens may shape the portion of the beam that is transmitted to the perforation site.

The main receptacle 82 is formed by the lens and a wall 88, preferably extending essentially away from the perimeter of the lens. The open end of the main receptacle or rim 90 is placed adjacent to the perforation site. The area defined by the lens, wall of the main receptacle and perforation site is thereby substantially enclosed during the operation of the laser perforator device.

The base 86 is the part of the container unit that can optionally be inserted into the applicator. The base may comprise a cylinder, a plurality of prongs or other structure. The base may optionally have threading. Optionally, the base, when fully inserted, disengages a safety mechanism of the laser perforator device, allowing the emission of the laser beam.

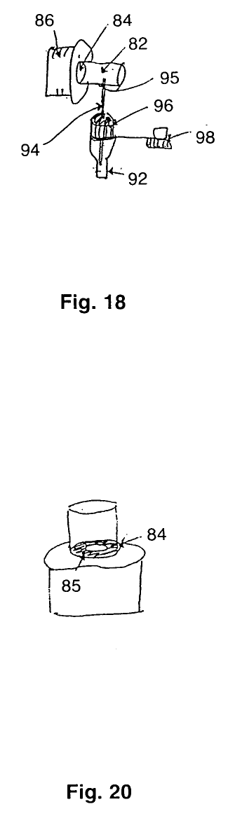

As shown in FIG. 18, the container unit may also include an additional vessel 92 which collects a portion of the matter released as part of the perforation. For example, this vessel can collect blood and/or other liquid or particulate matter, while the main receptacle 82 collects the ablated tissue. The blood and/or other liquid or particulate matter may be channeled into the vessel through a capillary tube 94 or other tubing which extends from the main receptacle into the vessel. The vessel is optionally detachable. The main receptacle may have a hole 95 in the wall through which the capillary tube or other tubing may be securely inserted. The vessel may have a removable stop 96 which sufficiently covers the open end of the vessel to prevent contamination with undesired material, but has an opening large enough for the capillary tube or other tubing to be inserted. In the preferred embodiment, the capillary tube or other tubing will extend outwardly from the main receptacle's wall and into the vessel through the removable stop. Once the sample has been collected, the stop may optionally be removed and discarded. The vessel may then optionally be sealed with a cap 98 to prevent spillage. The vessel is preferably bullet shaped.

Additionally, the interiors of the main receptacle 82, the capillary tube 94 or other tubing, and/or the additional vessel 92 are optionally coated with anticoagulating and/or preservative chemicals. Examples of preservatives include ethylenediaminetetraacetic acid (EDTA) or sodium benzoate. Examples of anticoagulating chemicals are sodium heparin and sodium citrate.

In the first embodiment, the container unit comprises a cylindrical main receptacle 82, a cylindrical base 86, and an at least partially transparent circular lens 84 in the area between the main receptacle and base. Optionally, the lens may include a mask which shapes the beam that perforates the tissue. The interior of the main receptacle is optionally coated with anticoagulating and/or preservative chemicals. The container unit is constructed of glass or plastic. The container unit is optionally disposable.

In the second embodiment, the container unit comprises the elements of the first embodiment and also includes an additional vessel 92 and a capillary tube 94 extending outwardly from the main receptacle's wall 88 and into the vessel through a removable stop 96. The vessel may optionally have a cap 98 to seal the opening so as to prevent spillage. The interior of the main receptacle, the capillary tube, and/or the additional vessel may optionally be coated with anticoagulating and/or preservative chemicals. The container unit is constructed of glass or plastic. The container unit, including the capillary tube and the additional vessel, are optionally disposable.

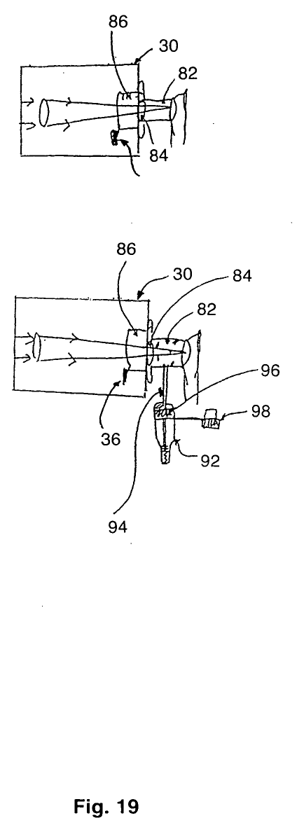

FIG. 19 shows examples of the use of the container unit with the laser perforator device. In this embodiment the applicator 30 is surrounded by the housing 34. The container unit is inserted in the applicator 30 and aligned so as to be capable of defeating the interlock 36. The base 86 of the container unit in this embodiment is within the applicator 30, while the rim 90 of the receptacle 82 is located adjacent to the tissue to be perforated. Optionally the additional vessel 92 can be connected by tubing 94 to the receptacle. The beam passes through the lens 84.

In a third embodiment, the container unit is evacuated. The optional vacuum in the container unit exerts a less than ambient pressure over the perforation site, thereby increasing the efficiency in blood collection. The container unit is optionally coated with anticoagulating and/or preservative chemicals. The container unit's end proximal to the perforation site is optionally sealed air-tight with a plug 70. The plug is constructed of material of suitable flexibility to conform to the contours of the perforation site (e.g., the finger). The desired perforation site is firmly pressed against the plug. The plug's material is impermeable to gas transfer. Furthermore, the plug's material is thin enough to permit perforation of the material as well as perforation of the skin by the laser. In the preferred embodiment, the plug is constructed of rubber.

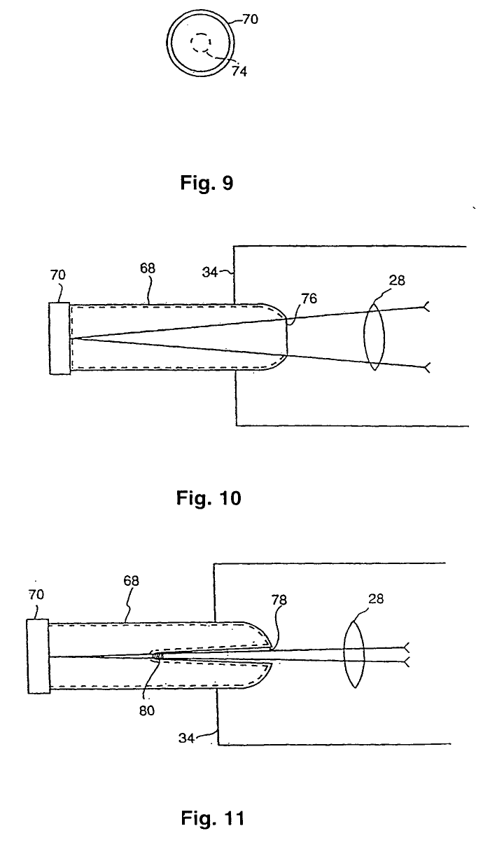

The plug perforation center 74, as shown in FIG. 9, is preferably constructed of a thin rubber material. The thickness of the plug is such that the plug can maintain the vacuum prior to perforation, and the laser can perforate both the plug and the tissue adjacent to the plug. For use with an Er:YAG laser, the plug should be in the range of approximately 100 to 500 microns thick, but at the most 1 millimeter thick.

The plug perforation center 74 is large enough to cover the perforation site. Optionally, the perforated site is a round hole with an approximate diameter ranging from 0.1-1 mm, or slit shaped with an approximate width of 0.05-0.5 mm and an approximate length up to 2.5 mm. Thus, the plug perforation center is sufficiently large to cover perforation sites of these sizes.

The perforation site is firmly pressed against the rubber material. Optionally, an annular ring of adhesive can be placed on the rubber plug to provide an air-tight seal between the perforation site and the container unit. Preferably the perforation site on the plug is stretched when the tissue is pressed against the plug. This stretching of the plug material causes the hole created in the plug to expand beyond the size of the hole created in the tissue. As a result, the blood and/or serum can flow unimpeded into the container unit 68. The laser beam penetrates the container unit, perforates the plug perforation center 74 and perforates the patient's tissue.

In a fourth embodiment of the container unit, as shown in FIG. 10, the container unit 68 includes a hole 76 through which the laser passes. In this fourth embodiment, the container unit optionally solely collects ablated tissue. As in the other embodiments, the perforation site is firmly pressed against the container unit. The container unit can optionally include a plug proximal to the perforation site, however it is not essential because there is no need to maintain a vacuum in this embodiment. All embodiments of the container unit reduce the noise created from interaction between the laser beam and the patient's tissue and thus alleviate the patient's anxiety and stress.

Optionally, the container unit is disposable, so that the container unit and plug can be discarded after use. Additionally, the main receptacle of the container unit, capillary tube and/or additional vessel can contain reagents for various tests to be performed on the collected blood. Examples of such reagents are sodium heparin and other reagents known in the art to be used in standard blood chemistry tests (Garza, D. et al., Phlebotomy Handbook (3d edition), Appleton and Lang Pub. Co., Norwalk, Conn.; 1993, which is incorporated herein by reference). The reagents are positioned so that they will not be in the pathway of the laser light. The reagents are preferably present in a dry form, coating the interior walls of the collection part of the container unit, and thus readily available for interaction with the blood sample as it is collected.

A preferable configuration for the container unit when it contains a reagent is shown in FIG. 11. In this configuration, the container unit has an indentation 78 at the base such that any fluid reagent present in the container unit will not fall into the line of fire of the laser beam when the container unit is held either vertically or horizontally. The apex 80 of the indented area is made of an infrared-transparent substance such as quartz.

When reagents are present in the container unit prior to collection of the blood sample, it is beneficial to label the container unit in some manner as to the reagents contained inside, or as to the test to be performed on the sample using those reagents. A preferred method for such labelling is through the use of color-coded plugs. For example, a blue plug might indicate the presence of reagent A, while a red plug might indicate the presence of reagents B plus C within the container unit.

In order to sterilize the skin before perforation, a sterile alcohol-impregnated patch of paper or other thin material can optionally be placed over the site to be perforated. This material can also prevent the blowing off of potentially infected tissue in the plume released by the perforation. The material must be transparent to the laser beam. Examples of such material are a thin layer of quartz, mica, or sapphire. Alternatively, a thin layer of plastic, such as a film of polyvinyl chloride, can be placed over the skin. Although the laser beam will perforate the plastic, the plastic prevents most of the plume from flying out and thus decreases any potential risk of contamination from infected tissue. Additionally, a layer of a viscous sterile substance such as vaseline can be added to the transparent material or plastic film to increase adherence of the material or plastic to the skin and further decrease plume contamination. Additionally, such a patch can be used to deliver allergens, local anesthetics or other pharmaceuticals as described below.

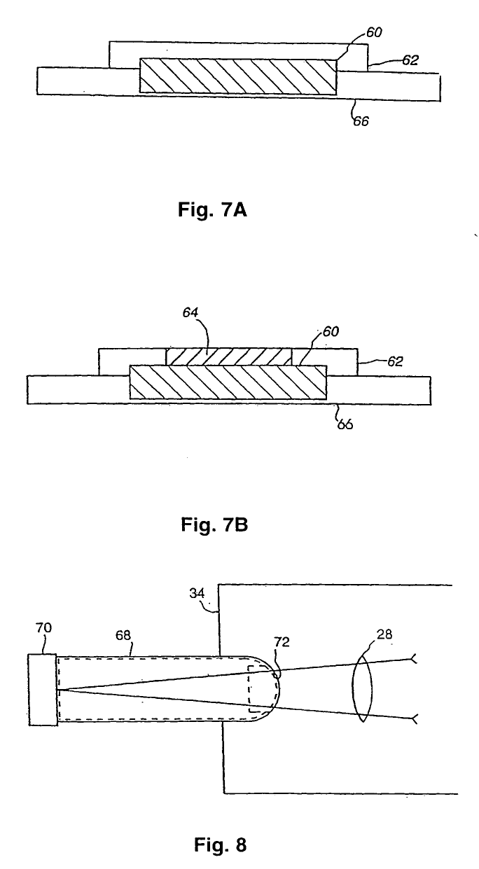

Examples of such a patch are provided in FIGS. 6 and 7A-7B. In FIG. 6, alcohol impregnated paper 54 is surrounded by a temporary adhesive strip 58. Side views of two alternative patches are shown in FIG. 7, where a sterilizing alcohol, antibiotic ointment, allergen, or pharmaceutical is present in the central region of the patch 60. This material is held in place by a paper or plastic layer 62, optionally with a laser-transparent material 64 such as mica, quartz or sapphire which is transparent to the laser beam at the center of the patch. The patch can be placed on the skin using an adhesive 66.

Factors which should be considered in defining the laser beam are wavelength, energy fluence, pulse temporal width and irradiation spot-size. The wavelength is determined by the laser material, such as Er:YAG, used in the device. The pulse temporal width is a consequence of the pulse width produced by the bank of capacitors, the flashlamp, and the laser rod material. The pulse width is optimally between 1 and 1,000 microseconds. The laser beam is focused precisely on the skin, creating a beam diameter at the skin in the range of 0.1-1 mm, or optionally a slit-shaped beam ranging in width from 0.05 to 0.5 mm, and in length up to 2.5 mm. The energy density, which is a function of laser energy output (in Joules) and size of the beam at the focal point (cm.sup.2), should be in the range of 10-100,000 J/cm.sup.2. The focal length of the lens can be of any length, but in one embodiment of the device is 30 mm. The energy fluence rate is preferably in the range of 1.3.times.10.sup.4 to 6.4.times.10.sup.10 Watts/cm.sup.2 and concurrently the energy fluence rate is preferably in the range of 1.3.times.10.sup.1 to 6.4.times.10.sup.7 Watts/cm.sup.2.

The device operates as follows: The power interlock switch is initiated, thus starting the charging of the capacitors. The device is manipulated in such a way that a portion of the patients skin is positioned at the site of the laser focus within the applicator. For blood collection, the location of the perforation is optimally at a site where the blood flow is high. Examples of such regions of the skin are on a fingertip, or the heel of a foot. For perforation for delivery of anesthetics or pharmaceuticals or for immunization, a region of the skin which has less contact with hard objects or with sources of contamination is preferred. Examples are skin on the arm, leg, abdomen or back. Optionally, the skin heating element is activated at this time.

Preferably a holder is provided with a hole coincident with the focal plane of the optical system. Optionally, a spring-loaded interlock 36 can be attached to the holder, so that when the patient applies a small amount of pressure to the interlock, to recess it to the focal point, a switch is closed and the laser will initiate a pulse of radiation. In this setup, the focal point of the beam is not in line with the end of the holder, until that end is depressed. In the extremely unlikely event of an accidental discharge of the laser before proper positioning of the tissue at the end of the laser applicator, the optical arrangement will result in an energy fluence rate that is significantly low, thus causing a negligible effect on unintentional targets.

For certain purposes, it is useful to create multiple perforations of the skin simultaneously or in rapid sequence. To accomplish this, a beam-splitter can optionally be added to the device.

Drawing Blood or Serum

The device can be used to perforate the skin to the capillary layer to allow the collection of blood. The blood can be used for a wide variety of tests, such as to determine blood chemistry (blood sugar, BC, urea, electrolytes, creatinine, cholesterol, etc.), and/or it can be fractionated into its components, such as serum and cells, for a variety of purposes, such as determination of red blood cell count. The blood can also be used for such purposes as genetic analysis for genetic counseling.

With the other parameters set, the intensity of the laser pump source will determine the intensity of the laser pulse, which will in turn determine the depth of the resultant perforation. Therefore, various settings on the device can be provided to allow penetration of different thicknesses of skin.

As described above, the skin can be preheated to dilate the capillaries and increase the blood flow before perforation. This increased blood flow allows a greater volume of blood to be collected and obviates the need for multiple perforations. Preheating can be accomplished by the addition of a preheater, as described above, or by other means of preheating the skin prior to positioning it on the laser applicator part of the device.

Optionally, a beam-dump is positioned in such a way as not to impede the use of the laser for puncturing fingertips. The beam-dump will absorb any stray electromagnetic radiation from the beam which is not absorbed by the tissue, thus preventing any scattered rays from causing damage. The beam-dump is easily removable for situations when the presence of the beam-dump would impede the placement of a body part on the applicator.

This method of drawing blood creates a very small zone in which tissue is vaporized, and only an extremely small zone of thermal necrosis. A practical round hole can range from 0.1-1 mm in diameter, while a slit shaped hole can range from approximately 0.05-0.5 mm in width and up to approximately 2.5 mm in length. As a result, healing is quicker or as quick as the healing after a skin puncture with a sharp implement.

The blood can be collected into a suitable vessel, such as a small test tube or a capillary tube, or in a container unit placed between the laser and the tissue as described above. The laser of this invention is particularly suited to collection of blood because it does not coagulate the blood upon penetration of the skin. Furthermore, the process is non-contact and so neither the patient, the blood to be drawn, or the instrument creating the perforation is contaminated.

Delivery of Pharmaceuticals

By appropriate modification of the power level, and/or the spot size of the laser beam, perforations can be made which do not penetrate the skin as deeply as described above. These perforations can be made through only the outer surfaces, such as the keratin layer or both the keratin layer and the epidermis. Optionally an optical beam-splitter can be employed so that either single perforations or a number of perforations within a desired area can be made. After perforation, the pharmaceutical can be applied to the skin in a formulation such as a cream, lotion or patch.

Immunization

As for delivery of pharmaceuticals, antigens can be administered through the skin for immunization purposes. The perforations are made through the outer layers of the skin, either singly or multiply, and the immunogen is provided in an appropriate formulation. For booster immunizations, where delivery over a period of time increases the immune response, the immunogen can be provided in a formulation which penetrates slowly through the perforations, but at a rate faster than possible through unperforated skin.

Delivery of Anesthesia

Localized anesthetics can be delivered using the method and device of this invention. Topically applied anesthetics must penetrate the keratin layer in order to be effective. Presently, compounds acting as drug carriers are used to facilitate the transdermal diffusion of some drugs. These carriers sometimes alter the behavior of the drug, or are themselves toxic. The energy level on the device can be set appropriately to penetrate the keratin layer without penetrating to the capillary layer. Anesthetic can then be applied to the perforations, for example in an impregnated patch.

Delivery of Allergens

This device and method can also be applied to the delivery of allergens for example for allergy testing. Multiple perforations can be made, through the outer layer of the skin, but not penetrating to the capillary level. A variety of allergens can then be applied to the skin, as in a skin patch test.

The following examples are descriptions of the use of the device of this invention for the purpose of drawing blood. These examples are not meant to limit the scope of the invention, but are merely one embodiment.

### EXAMPLE 1

An infrared laser radiation pulse was formed using a solid state, pulsed, multimode Er:YAG laser consisting of two resonator mirrors, an Er:YAG crystal as an active medium, a power supply, and a means of focusing the laser beam. The wavelength of the laser beam was 2.94 microns. The duration of the pulse was approximately 100 microseconds. The elliptical spot size was approximately 0.2-0.3 by 1-2 mm. Impulse energy used was 0.7, 0.9 or 2.0 J for thin to thick skin respectively. Single pulses were used, but in one test, 6 pulses per minute were used, each irradiating a-separate piece of tissue.

The operating parameters were as follows: The energy per pulse was 2 Joules, with the size of the beam at the focal point being 0.2 mm, creating an energy fluence of 10.sup.3 J/cm.sup.2. The pulse temporal width was 100 microseconds, creating an energy fluence rate of 1.times.10.sup.7 Watts/cm.sup.2.

Each patient's finger was treated prior to perforation with 96% ethyl alcohol to remove bacteria. The finger was placed at the focal point of the laser, and the laser was discharged. The blood was drawn from the perforation with a glass capillary tube. The volume of blood drawn (with no squeezing of the finger) ranged from 0.5-1.0 ml. This blood did not differ in chemistry from comparable samples obtained by lancet puncture during control tests. The pain elicited by laser perforation was estimated to be equal to or less than the pain elicited by the stabbing puncture of a lancet.

Morphological analysis of the effect of the laser perforation on the skin tissue showed minimal area of thermal destruction (less than 20-40 microns beyond the edge of the perforation produced) without any signs of carbonization. The wounds were cone shaped. The depth and width of the wounds were found to be proportional to the energy fluence and were approximately related to the inverse duration of the laser pulse.

### EXAMPLE 2

The laser perforator comprises a flashlamp (PSC Lamps, Webster, N.Y.), an Er:YAG crystal (Union Carbide Crystal Products, Washagoul, Wash.), optical-resonator mirrors (CVI Laser Corp., Albuquerque, N. Mex.), an infrared transmitting lens (Esco Products Inc., Oak Ridge, N.J.), as well as numerous standard electrical components such as capacitors, resistors, inductors, transistors, diodes, silicon-controlled rectifiers, fuses and switches, which can be purchased from any electrical component supply firm, such as Newark Electronics, Little Rock, Ark.

### EXAMPLE 3

An infrared laser radiation pulse was formed using a solid state, pulsed, multimode Er:YAG laser consisting of two flat resonator mirrors, an Er:YAG crystal as an active medium, a power supply, and a means of focusing the laser beam. The wavelength of the laser beam was 2.94 microns. The duration of the pulse was approximately 100 microseconds. The elliptical spot size was approximately 0.2-0.3 by 1-2 mm. Impulse energy used was 0.7, 0.9 or 2.0 J for thin to thick skin respectively. Single pulses were used, but in one test, 6 pulses per minute were used, each irradiating a separate piece of tissue.

The operating parameters were as follows: The energy per pulse was 2 Joules, with the size of the beam at the focal point being 0.2 mm by 1 mm, creating an energy fluence of 10.sup.3 J/cm.sup.2. The pulse temporal width was 100 microseconds, creating an energy fluence rate of 1.times.10.sup.7 Watts/cm.sup.2.

Each patient's finger was treated prior to perforation with 96% ethyl alcohol to remove bacteria. The finger was placed at the focal point of the laser, and the laser was discharged. The blood was drawn from the perforation with a glass capillary tube. The volume of blood drawn (with no squeezing of the finger) ranged from 0.5-1.0 ml. This blood did not differ in chemistry from comparable samples obtained by lancet puncture during control tests. The pain elicited by laser perforation was estimated to be less than the pain elicited by the stabbing puncture of a lancet.

Morphological analysis of the effect of the laser perforation on the skin tissue showed minimal area of thermal destruction (less than 20-40 microns beyond the edge of the perforation produced) without any signs of carbonization. The wounds were slit shaped. The depth and width of the wounds were found to be proportional to the energy fluence and were approximately related to the inverse duration of the laser pulse.

### EXAMPLE 4

Perforation is performed as in Example 1 or 3 except that the device is modified to include a blood collection tube, fitted tightly between the front end of the laser device and the focal point of the laser, through which the laser beam passes. The tube is 2.0 cm in. length and 1.0 cm in diameter, with an indentation in the bottom which pushes the bottom 1.0 cm into the center of the tube. As a result, any fluid or crystallized additive such as the anticoagulant sodium heparin will not fall into the line of fire of the laser beam when the tube is held either vertically or horizontally. The apex of the indented area is made of a quartz disc which is transparent to the laser beam.

The distal end of the tube is covered by a rubber plug. The plug is coated on the outside with an adhesive to cause adherence of the plug to the skin to be perforated. The tube itself is maintained with an interior vacuum prior to perforation. The tube is further coated on the inside with sodium heparin to act as an anticoagulant for purposes of performing a blood count on the sample obtained.

The laser is then fired, causing the laser beam to pass through the tube, perforating only the distal end (the plug) of the tube as well as the skin. A specimen of blood of approximately 1 cc will then flow into the tube and mix with the sodium heparin. All of the blood specimen as well as any exploded/ablated tissue is thus contained within the tube, preventing contamination and the spread of disease.

While embodiments and applications of this invention have been shown and described, it would be apparent to those skilled in the art that many more modifications are possible without departing from the inventive concepts herein. The invention, therefore, is not to be restricted except in the spirit of the appended claims.

1. A method of introducing a substance into a living body, comprising:
forming a plurality of areas on the stratum corneum of the skin of a living body having enhanced permeability through to the capillary layer by generating a single laser beam to irradiate the areas on the skin with subablative laser energy without substantially ablating the skin; and
introducing the substance into the body by bringing the substance in contact with the areas of enhanced permeability.

2. The method of claim 1, wherein forming the plurality of areas comprises;
passing the single laser beam through a beam splitter to split the single laser beam into a plurality of laser beams to form the plurality of areas substantially concurrently.

3. The method of claim 2, wherein the single laser beam is generated with a laser selected from the group consisting of Er:YAG, pulsed CO², Ho:YAG, Er/Cr:YSSG, Ho:YSSG, Er:GGSG, Er:YLF, Tm:YAG, Ho/Nd:Ya 10₃, cobalt:MgF₂, HF chemical, DF chemical, carbon monoxide, deep UV laser, diode laser, frequency tripled Nd:YAG, and combinations thereof.

4. The method of claim 3, wherein generating the single laser beam comprises:
generating the single laser beam to irradiate the skin with subablative laser energy having a wavelength of about 2 to about 7 microns, an energy fluence of about 10 to about 100,000 J/cm², and a target area on the skin of about 0.1 to about 1 mm in diameter.

5. The method of claim 3, wherein generating the single laser beam comprises:
generating the single laser beam to irradiate the skin with subablative laser energy having a wavelength of approximately 2.94 microns.

6. The method of claim 2, wherein the beam splitter is selected from the group consisting of a series of partially silvered mirrors, a series of dichroic mirrors, and a series of beam-splitting prisms.

7. The method of claim 1, wherein forming the plurality of areas comprises:
passing the single laser beam through an acousto-optic modulator to sequentially deflect the laser beam at a plurality of angles to form each of the plurality of areas.

8. The method of claim 7, wherein the single laser beam is generated with a laser selected from the group consisting of Er:YAG, pulsed CO², Ho:YAG, Er/Cr:YSSG, Ho:YSSG, Er:GGSG, Er:YLF, Tm:YAG, Ho/Nd:Ya 10₃, cobalt:MgF₂, HF chemical, DF chemical, carbon monoxide, deep UV laser, diode laser, frequency tripled Nd:YAG, and combinations thereof.

9. The method of claim 8, wherein generating the single laser beam comprises:
generating the single laser beam to irradiate the skin with subablative laser energy having a wavelength of about 2 to about 7 microns, an energy fluence of about 10 to about 100,000 J/cm², and a target area on the skin of about 0.1 to about 1 mm in diameter.

10. The method of claim 9, wherein generating the single laser beam comprises:
generating the single laser beam to irradiate the skin with subablative laser energy having a wavelength of approximately 2.94 microns.
